(19) United States
(12) Patent Application Publication
(10) Pub. No.: US 2006/0111428 A1
(43) Pub. Date: May 25, 2006

(54) Combination of an dpp-iv inhibitor and a ppar-alpha compound

(76) Inventor: Pei-Ran Wang, Cambridge, MA (US)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 10/537,870

(22) PCT Filed: Dec. 9, 2003
(86) PCT No.: PCT/EP03/13963

§ 371 Date: Aug. 30, 2005

Related U.S. Application Data
(60) Provisional application No. 60/432,192, filed on Dec. 10, 2002.

(51) Int. Cl. A61K 31/4025; A61K 31/401

(52) U.S. Cl. 514/422; 514/423

(57) ABSTRACT

The invention relates to a combination which comprises a DPP-IV inhibitor and at least one further PPARα compound, preferably selected from the group consisting of fenofibrate, micronized fenofibrate, bezafibrate, gemfibrazil and ciprofibrate for simultaneous, separate or sequential use in the prevention, delay of progression or treatment of conditions mediated by DPP-IV and PPARα, in particular diabetes, more especially type 2 diabetes mellitus, conditions of IGT, conditions of impaired fasting plasma glucose, metabolic acidosis, ketosis, arthritis, obesity, dyslipidemia and osteoporosis.

The invention relates to a combination, such as a combined preparation or pharmaceutical composition, respectively, which comprises a dipeptidylpeptidase-IV (DPP-IV) inhibitor and a peroxisome proflierator-activated receptor a (PPARα), for simultaneous, separate or sequential use, especially in the prevention, delay of progression or treatment of conditions mediated by DPP-IV, in particular diabetes, more particular type 2 diabetes mellitus, conditions of impaired glucose tolerance (IGT), conditions of impaired fasting plasma glucose, metabolic acidosis, ketosis, arthritis, obesity, dyslipidemia and osteoporosis; the use of such combination for the preparation of a pharmaceutical preparation for the prevention, delay of progression or treatment of such conditions; the use of such combination for the cosmetic treatment of a mammal in order to effect a cosmetically beneficial loss of body weight; a method of prevention, delay of progression or treatment of conditions mediated by DPP-IV; a method of improving the bodily appearance of a warm-blooded animal.

DPP-IV is responsible for inactivating GLP-1. More particularly, DPP-IV generates a GLP-1 receptor antagonist and thereby shortens the physiological response to GLP-1. GLP-1 is a major stimulator of pancreatic insulin secretion and has direct beneficial effects on glucose disposal.

Non-insulin dependent diabetes mellitus (type 2 diabetes mellitus) is characterized by both increased peripheral insulin resistance and abnormal insulin secretion. At least three abnormalities of insulin secretion are recognized: in the first phase, insulin secretion is lost and in the second phase insulin is both delayed and inadequate in the face of elevated circulating glucose levels. Several metabolic, hormonal, and pharmacological entities are known to stimulate insulin secretion including glucose, amino-acids and gastrointestinal peptides. The Diabetes Control and Complications Trial (DCCT) has established that lowering of blood glucose is associated with decreases in the onset and progression of diabetic microvascular complications (see Diabetes Control and Complications Trial Research Group, N. Engl. J. Med., Vol. 329, pp. 977-986 (1993)). IGT is an impairment of glucose homeostasis closely related to type 2 diabetes mellitus. Both conditions convey a great risk of macrovascular disease. Therefore, one therapeutic focus is on optimizing and potentially normalizing glycemic control in subjects with type 2 diabetes mellitus, conditions of impaired fasting plasma glucose or IGT. Presently available agents need to be improved in order to better meet this therapeutic challenge.

The present invention relates to a combination which comprises a DPP-IV inhibitor in free or pharmaceutically acceptable salt form, and a PPARα or the pharmaceutically acceptable salt of such a compound and optionally at least one pharmaceutically acceptable carrier; for simultaneous, separate or sequential use.

Preferably, the PPARα compound is a fibrate selected from the group consisting of fenofibrate, micronized fenofibrate, bezafibrate, gemfibrazil and ciprofibrate, or the pharmaceutically acceptable salts of such a compound and optionally at least one pharmaceutically acceptable carrier; for simultaneous, separate or sequential use, particularly in the prevention, delay of progression or treatment of conditions mediated by DPP-IV or PPARα, in particular conditions of IGT, conditions of impaired fasting plasma glucose, metabolic acidosis, ketosis, arthritis, obesity and osteoporosis, and preferably diabetes, especially type 2 diabetes mellitus and dyslipidemia. Such a combination is preferably a combined preparation or a pharmaceutical composition.

The DPP-IV inhibitor can be peptidic or non-peptidic. Preferably, the DPP-IV inhibitor is non-peptidic.

In the present context “a DPP-IV inhibitor” is also intended to comprise active metabolites and prodrugs thereof, such as active metabolites and prodrugs of DPP-IV inhibitors. A “metabolite” is an active derivative of a DPP-IV inhibitor produced when the DPP-IV inhibitor is metabolised. A “prodrug” is a compound that is either metabolised to a DPP-IV inhibitor or is metabolised to the same metabolite(s) as a DPP-IV inhibitor.

DPP-IV inhibitors are known in the art. In the following reference is made to representatives of DPP-IV inhibitors:

Unless stated otherwise in the present disclosure organic radicals designated “lower” contain not more than 7, preferably not more than 4, carbon atoms and the following expressions have the meanings as given below:

Halogen represents preferably fluoro, chloro or bromo.

Lower alkyl is, if not stated otherwise, preferably ethyl or, most preferably, methyl. (C₁₋₈)Alkyl is branched or preferably unbranched alkyl, preferably lower alkyl, e.g., methyl or ethyl.

Lower alkylene is preferably methylene, ethylene or propylene. It can be unsubstituted or substituted, e.g., by hydroxy.

Lower alkoxy is preferably methoxy or ethoxy. (C₂₋₄)Alkoxy is, e.g., ethoxy or propoxy.

Cycloalkyl is, e.g., (C₃₋₁₂)cycloalkyl, preferably cyclopropyl, cyclobutyl, cyclopentyl, cyclohexyl, cycloheptyl or cyclodecyl; or bicycloalkyl, such as bicycloheptyl. Cycloalkenyl is preferably 1-cyclohexenyl, 2-cyclohexenyl, 3-cyclohexenyl, 1-cyclopentenyl or 1-cyclopentenyl.

(C₁₋₃)Hydroxyalkyl is, e.g., 3-hydroxypropyl, 1-hydroxyethyl or hydroxymethyl.

(C₄₋₆)Alkylenimino which is unsubstituted or substituted by one or two lower alkyl groups is, e.g., pyrrolidinyl, methylpyrrolidinyl, 1-piperidinyl, 2-piperidinyl, 3-piperidinyl, 2-methyl-1-piperidinyl or hexamethylenimino. Preferably, (C₄₋₆)alkylenimino is 1-piperidinyl.

A [3.1.1]bicyclic carbocyclic moiety optionally substituted as defined above preferably is bicyclo[3.1.1]hept-2-yl optionally disubstituted in 6-position with methyl, or bicyclo[3.1.1]-hept-3-yl optionally tri-substituted with one methyl in 2-position and two methyl groups in 6-position. A [2.2.1]bicyclic carbocyclic moiety optionally substituted as defined above preferably is bicyclo[2.2.1]hept-2-yl.

Aryl comprises preferably 6-12 carbon atoms and is, e.g., phenyl, tolyl or naphthyl, each of which can be substituted, e.g., by lower alkyl or halogen.

The term “heteroaryl” refers to an aromatic heterocyclic radical selected, e.g., from the group consisting of pyrrolidinyl, pyrrolyl, pyrazolyl, oxetanyl, pyrazolinyl, imidazolyl, imidazolinyl, imidazolidinyl, oxazolyl, oxazolidinyl, isoxazolinyl, isoxazolyl, thiazolyl, thiadiazolyl, thiazolidinyl, isothiazolyl, isothiazolidinyl, furyl, tetrahydrofuryl, thienyl, oxadiazolyl, piperidinyl, piperazinyl, azepinyl, 4-piperidinyl, pyridyl, pyrazinyl, pyrimidinyl, pyridazinyl, tetrahydropyranyl, morpholinyl, thiamorpholinyl, thiamorpholinyl sulfoxide, thiamorpholinyl sulfone, 1,3-dioxolane, indolyl, benzothiazolyl, benzoxazolyl, benzothienyl, quinuclidinyl, quinolinyl, tetrahydroisoquinolinyl, isoquinolinyl, benzimidazolyl, benzopyranyl, indolizinyl, benzofuryl, chromonyl, coumarinyl, benzopyranyl, cinnolinyl, quinoxalinyl, indazolyl, pyrrolopyridyl, furopyridinyl, dihydrobenzoisothiazolyl, dihydroisoindolyl, dihydroquinazolinyl and tetrahydroquinazolinyl.

Preferred DPP-IV inhibitors are N-(N′-substituted glycyl)-2-cyanopyrrolidines represented by formula (I), 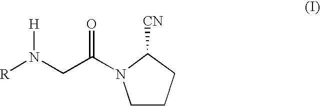
wherein R is:
a) R₁R₁ₐN(CH₂)ₘ—,
   wherein
      R₁ is a pyridinyl or pyrimidinyl moiety optionally mono- or, independently, di-substituted with lower alkyl, lower alkoxy, halogen, trifluoromethyl, cyano or nitro; or phenyl optionally mono- or, independently, di-substituted with lower alkyl, lower alkoxy or halogen;
      R₁ₐ is hydrogen or (C₁₋₈)alkyl; and
      m is 2 or 3;
b) (C₃₋₁₂)Cycloalkyl optionally mono-substituted in the 1-position with (C₁₋₃)hydroxyalkyl;
c) R₂(CH₂)ₙ—,
   wherein either
      R₂ is phenyl optionally mono- or, independently, di- or, independently, tri-substituted with lower alkyl, lower alkoxy, halogen or phenylthio optionally mono-substituted in the phenyl ring with hydroxymethyl; or is (C₁₋₈)alkyl; a [3.1.1]bicyclic carbocyclic moiety optionally mono- or pluri-substituted with (C₁₋₈)alkyl; a pyridinyl or naphthyl moiety optionally mono- or, independently, di-substituted with lower alkyl, lower alkoxy or halogen; cyclohexene; or adamantyl; and
      n is 1-3; or
      R₂ is phenoxy optionally mono- or, independently, di-substituted with lower alkyl, lower alkoxy or halogen; and
      n is 2 or 3;
d) (R₃)₂CH(CH₂)₂—, wherein each R₃, independently, is phenyl optionally mono- or, independently, di-substituted with lower alkyl, lower alkoxy or halogen;
e) R₄(CH₂)ₚ—,
   wherein
      R₄ is 2-oxopyrrolidinyl or (C₂₋₄)alkoxy; and
      p is 2-4;
f) isopropyl optionally mono-substituted in 1-position with (C₁₋₃)hydroxyalkyl;
g) R₅, wherein R₅ is indanyl, a pyrrolidinyl or piperidinyl moiety optionally substituted with benzyl, a [2.2.1]- or [3.1.1]bicyclic carbocyclic moiety optionally mono- or pluri-substituted with (C₁₋₈)alkyl, adamantyl or (C₁₋₈)alkyl optionally mono- or, independently, pluri-substituted with hydroxy, hydroxymethyl or phenyl optionally mono- or, independently, di-substituted with lower alkyl, lower alkoxy or halogen;
h) a substituted adamantyl
   in free form or in acid addition salt form.

In a preferred embodiment of the invention, the N-(N′-substituted glycyl)-2-cyanopyrrolidine is represented by formula (I),

wherein

R is R₁R₁ₐN(CH₂)ₘ—, wherein
   R₁ is a pyridinyl or pyrimidinyl moiety optionally mono- or, independently, di-substituted with lower alkyl, lower alkoxy, halogen, trifluoromethyl, cyano or nitro; or phenyl optionally mono- or, independently, di-substituted with lower alkyl, lower alkoxy or halogen;
   R₁ₐ is hydrogen or (C₁₋₈)alkyl; and
   m is 2 or 3;
      in free form or in acid addition salt form.

More preferably, the N-(N′-substituted glycyl)-2-cyanopyrrolidine is represented by formula (I),

wherein

R is R₁R₁ₐN(CH₂)ₘ—, wherein
   R₁ is a pyridinyl moiety optionally mono- or, independently, di-substituted with lower alkyl, lower alkoxy, halogen, trifluoromethyl, cyano or nitro;
   R₁ₐ is hydrogen or (C₁₋₈)alkyl; and
   m is 2 or 3;
      in free form or in acid addition salt form.

Most preferably, the N-(N′-substituted glycyl)-2-cyanopyrrolidine of formula (I) is (S)-1-{2-[5-cyanopyridin-2-yl)amino]ethyl-aminoacetyl}-2-cyano-pyrrolidine (DPP728) or (S)-1-[(3-hydroxy-1-adamantyl)amino]acetyl-2-cyano-pyrrolidine (LAF237) as described in WO01/52825.

In another preferred embodiment, the DPP-IV inhibitor is selected from the compounds of formulae (II), (III), (IV) and (V): 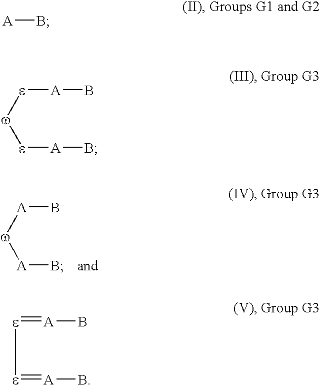
wherein 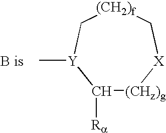
f is 1 or 2;
g is 0, 1 or 2;
X is CH₂, O, S, SO, SO₂, NH or NRα₁, where Rα₁ is lower alkyl (C₁₋₆);
—Y is —N, —CH or —C═ (when the —CO group of A is replaced with —CH═ or —CF═);
Rα is H, CN, CHO, B(OH)₂, PO₃H or an ester thereof, CC—Rα₇, or CH═N—Rα₈,
   wherein
      Rα₇ is H, F, lower alkyl (C₁₋₆), CN, NO₂, ORα₉, CO₂Rα₉ or CORα₉;
      Rα₉ is lower alkyl (C₁₋₆);
      Rα₈ is Ph, OH, ORα₉, OCORα₉ or OBn; and
      A is attached to Y;
         and wherein for the group G1 compounds
         (a) when Rα is H, A is an α-amino-acyl group derived from an α-amino-acid bearing a cycloaliphatic side-chain or is a β-amino-acyl group of general formula 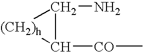
         wherein h is 1-6, the ring in either case optionally having unsaturation and/or heteroatom substitution;
         (b) when Rα is CN, CC—Rα₇ or CH═N—Rα₈, A is as defined at (a) and in addition may be derived from any L-α-amino acid bearing a lipophilic side-chain;
         (c) and when Rα is CHO or B(OH)₂, A is a β-amino-acyl group as defined under (a);
         for the group G2 compounds, Rα is H, CN, C═C—Rα₇ or —CH═N—Rα₈ and A is 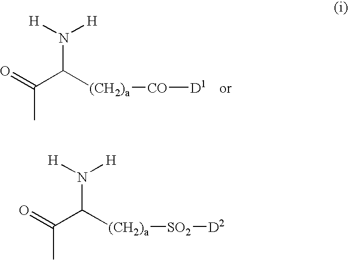
         wherein
a is 1-5;
D¹ is -G-(CH₂)_{b}—(Rα₄)_{q}—Rα₃, where
   G is O, NH or NMe;
   b is 0-12;
   q is 0-5;
   D² is D¹ with G≠O;
   Rα₄ is Z-NH—(CH₂)ₙ— or NH-Z-(CH₂)_{c}—,
   wherein
      c is 1-12; and
      Z is CO, CH₂ or SO₂;
   Rα₃ is CO₂H; or an ester thereof, CONH₂, CONHNH₂, CONRα₅Rα₆, CONHNRα₅Rα₆, PO₃H or an ester thereof, SO₃H, SO₂NH₂, SO₂NRα₅Rα₈, OH, ORα_{N}, substituted or unsubstituted aryl or heteroaryl, NH₂, NRα₅Rα₆, NHCO₂Rα₅, NHSO₂NRα₅Rα₆, NHCORα₅, NH—SO₂Rα₅, NH—CH(:NRα₅)NRα₅Rα₆, NHCONHRα₅Rα₆, sugar, CO-aminosugar, NHCO-aminosugar or —NHCS-aminosugar,
   wherein
      Rα₅ and Rα₆ are, independently, selected from H and lower alkyl, fluoroalkyl and cycloalkyl group of up to 8 atoms and aryl, heteroaryl and alkyl heteroaryl groups of up to 11 atoms or Rα₅ and Rα₆ may together comprise a chain (C₃₋₈); or is 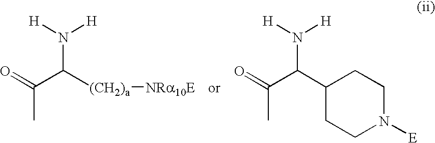
         wherein
Rα₁₀ is H or Me, the ring may contain more heteroatoms;
E is J-(CH₂)_{b}—(Rα₄)_{q}—Rα₃,
wherein
   J=CO, CH₂ or SO₂; and
   a, b, q, Rα₃ and Rα₄ are as defined under (i); or is 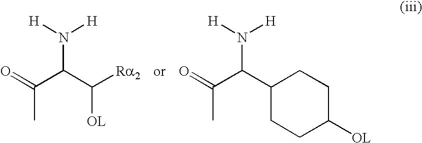
      wherein
Rα₂ is H or Me, the ring may contain one or more heteroatoms; and
L is (CH₂)_{d}—(CO)ᵣ—(CH₂)_{b}—(Rα₄)_{q}—Rα₃ or (CH₂)ₑ—NRα₁₀—(CH₂)_{b}—(Rα₄)_{q}—Rα₃,
wherein
   r is 0 or 1;
   d is 0-4;
   e is 2-4; and
   b, q, Rα₃ and Rα₄ are as defined under (i);
      and for the group G3 compounds, each B may have any Identity defined therefor above, each A may be chosen from any group G2 structure (i), (ii) or (iii) above with the terminal groups Rα₃ in the A residues replaced with a shared group -ε-ω-ε- or -ε-ε- or -ω-, and ε and ω are selected, independently, from CH₂, O, NH, CO, S, SO₂, Ph and NHMe; and wherein in groups G2 and G3 at least one CH₂ group in a chain may be replaced by a bioisostere thereof or any amide group which connects A and B in a group G 1, G2 or G3 compound or which is in a side chain of A in a Group G2 or G3 compound may be replaced by an amide bioisostere;
      in free form or in acid addition salt form.

DPP-IV inhibitors are in each case generically and specifically disclosed e.g. in WO 98/19998, DE19616 486 A1, WO 00/34241, WO 95/15309, WO 01/72290, WO01/52825, WO03/002553, WO 9310127, WO 99/61431, WO 9925719, WO 9938501, WO 9946272, WO 9967278 and WO 9967279. In each case in particular in the compound claims and the final products of the working examples, the subject matter of the final products, the pharmaceutical preparations and the claims are hereby incorporated into the present application by reference to these publications.

WO 9819998 discloses N-(N′-substituted glycyl)-2-cyano pyrrolidines, in particular 1-[2-[5-Cyanopyridin-2-yl]amino]-ethylamino) acetyl-2-cyano-(S)-pyrrolidine.

Preferred compounds described in WO03/002553 are listed on pages 9 to 11 and are incorporated into the present application by reference.

DE1 9616 486 A1 discloses val-pyr, val-thiazolidide, isoleucyl-thiazolidide, isoleucyl-pyrrolidide, and fumar salts of isoleucyl-thiazolidide and isoleucyl-pyrrolidide.

WO 0034241 and U.S. Pat. No. 6,110,949 disclose N-substituted adamantyl-amino-acetyl-2-cyano pyrrolidines and W (substituted glycyl)-4-cyano pyrrolidines respectively. DPP-IV inhibitors of interest are specially those cited in claims 1 to 4.

WO 9515309 discloses amino acid 2-cyanopyrrolidine amides as inhibitors of DPP-IV and WO 9529691 discloses peptidyl derivates of diesters of alpha-aminoalkylphosphonic acids, particularly those with proline or related structures. DPP-IV inhibitors of interest are specially those cited in Table 1 to 8.

In WO 01/72290 DPP-IV inhibitors of interest are specially those cited in example 1 and claims 1, 4, and 6.

WO01/52825 specially discloses (S)-1-{2-[5-cyanopyridin-2yl)amino]ethyl-aminoacetyl)-2-cyano-pyrrolidine or (S)-1-[(3-hydroxy-1 adamantyl)amino]acetyl-2-cyano-pyrrolidine.

WO 9310127 discloses proline boronic esters useful as DPP-IV inhibitors. DPP-IV inhibitors of interest are specially those cited in examples 1 to 19.

Published patent application WO 9925719 discloses sulphostin, a DPP-IV inhibitor prepared by culturing a Streptomyces microorganism.

WO 9938501 discloses N-substituted 4- to 8-membered heterocyclic rings. DPP-IV inhibitors of interest are specially those cited in claims 15 to 20.

WO 9946272 discloses phosphoric compounds as inhibitors of DPP-IV. DPP-IV inhibitors of interest are specially those cited in claims 1 to 23.

Other preferred DPP-IV inhibitors are the compounds of formula I, II or III disclosed in the patent application WO 03/057200 on page 14 to 27. Most preferred DPP-IV inhibitors are the compounds specifically described on pages 28 and 29.

Published patent applications WO 9967278 and WO 9967279 disclose DPP-IV prodrugs and inhibitors of the form A-B-C where C is either a stable or unstable inhibitor of DPP-IV.

Preferably, the N-peptidyl-O-aroyl hydroxylamine is a compound of formula VII 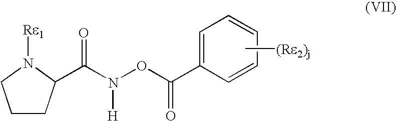
wherein
j is 0, 1 or 2;
Rε₁ represents the side chain of a natural amino acid; and
Rε₂ represents lower alkoxy, lower alkyl, halogen or nitro;
   or a pharmaceutically acceptable salt thereof.

In a very preferred embodiment of the invention, the N-peptidyl-O-aroyl hydroxylamine is a compound of formula VIIIa 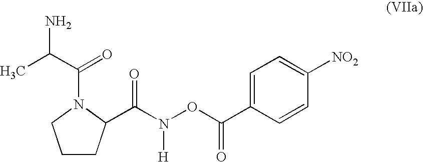
or a pharmaceutically acceptable salt thereof.

N-Peptidyl-O-aroyl hydroxylamines, e.g. of formula VIII or Vila, and their preparation are described by H. U. Demuth et al. in J. Enzyme Inhibition 1988, Vol. 2, pages 129-142, especially on pages 130-132.

Preferred DPP-IV inhibitors are those described by Mona Patel and col. (Expert Opinion Investig Drugs. 2003 April;12(4):623-33) on the paragraph 5, especially P32/98, K-364, FE-999011, BDPX, NVP-DDP-728 and others, which publication is hereby incorporated by reference especially the described DPP-IV inhibitors.

FE-999011 is described in the patent application WO 95/15309 page 14, as compound No. 18.

P32/98 (CAS number: 251572-86-8) also known as 3-[(2S,3S)-2-amino-3-methyl-1-oxopentyl]thiazolidine is a compound of formula (VI) 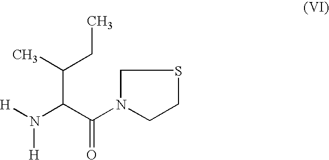
in free form or in acid addition salt form. Preferably P32/98 is used as 3-[(2S,3S)-2-amino-3-methyl-1-oxopentyl]thiazolidine and (2E)-2-butenedioate (2:1) mixture as described below 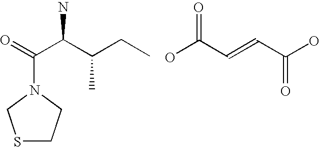
and is described in the patent application WO 99/61431.

DPP-IV inhibitors are in each case generically and specifically disclosed in WO 98/19998, DE 196 16 486 A1, WO 00/34241 and WO 95/15309, in each case in particular in the compound claims and the final products of the working examples, the subject-matter of the final products, the pharmaceutical preparations and the claims are hereby incorporated into the present application by reference to these publications. DPP728 and LAF237 are specifically disclosed in Example 3 of WO 98/19998 and Example 1 of WO 00/34241, respectively. A DPP-IV inhibitor of formula (VI) (see above) is specifically described in Diabetes, Vol. 47, pp. 1253-1258 (1998). DPP728 can be formulated as described on page 20 of WO 98/19998.

In a further preferred embodiment, the DPP-IV inhibitor is a N-peptidyl-O-aroyl hydroxylamine or a pharmaceutically acceptable salt thereof. Aroyl is, e.g., naphthylcarbonyl; or benzoyl which is unsubstituted or mono- or di-substituted, e.g., by lower alkoxy, lower alkyl, halogen or, preferably, nitro. The peptidyl moiety comprises preferably two α-amino acids, e.g., glycine, alanine, leucine, phenylalanine, lysine or proline, of which the one attached directly to the hydroxylamine nitrogen atom is preferably proline.

Preferably, the N-peptidyl-O-aroyl hydroxylamine is a compound of formula (VII) 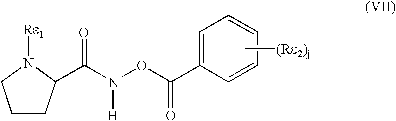
wherein
j is 0, 1 or 2;
Rε₁ represents the side chain of a natural amino acid; and
Rε₂ represents lower alkoxy, lower alkyl, halogen or nitro;
   or a pharmaceutically acceptable salt thereof.

Preferred DPP-IV inhibitors are N-substituted adamantyl-amino-acetyl-2-cyano pyrrolidines, N (substituted glycyl)-4-cyano pyrrolidines, N-(N′-substituted glycyl)-2-cyanopyrrolidines, N-aminoacyl thiazolidines, N-aminoacyl pyrrolidines, L-allo-isoleucyl thiazolidine, L-threo-isoleucyl pyrrolidine, and L-allo-isoleucyl pyrrolidine, 1-[2-[(5-cyanopyridin-2-yl) amino] ethylamino] acetyl-2-cyano-(S)-pyrrolidine and pharmaceutical salts thereof.

Especially preferred are 1-{2-[(5-cyanopyridin-2-yl) amino] ethylamino} acetyl-2-(S)-cyano-pyrrolidine (DPP728), of formula: 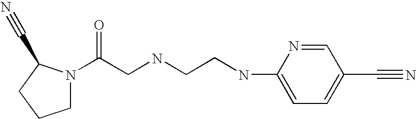
especially the dihydrochloride thereof;
pyrrolidine, 1-[(3-hydroxy-1-adamantyl) amino] acetyl-2-cyano-, (S) (LAF237) of formula 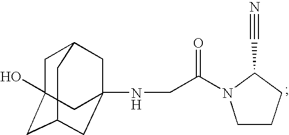
and L-threo-isoleucyl thiazolidine (compound code P32/98—see above), and pharmaceutical salts thereof.

In a very preferred embodiment of the invention, the N-peptidyl-O-aroyl hydroxylamine is a compound of formula (VIIa) 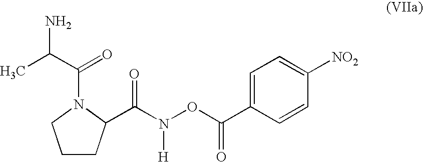
or a pharmaceutically acceptable salt thereof.

N-Peptidyl-O-aroyl hydroxylamines, e.g., of formula (VI) or (VIIa), and their preparation are described by Demuth et al., J. Enzyme Inhibition, Vol. 2, pp. 129-142 (1988), especially on pp. 130-132.

Any of the substances disclosed in the above mentioned patent documents, hereby included by reference, are considered potentially useful as DPP-IV inhibitors to be used in carrying out the present invention.

DPP-IV inhibitor to be used alone according to the present invention can be used in association with a carrier.

Comprised are likewise the corresponding stereoisomers as well as the corresponding polymorphs, e.g., crystal modifications, which are disclosed in the cited patent documents.

In a very preferred embodiment of the invention, the DPP-IV Inhibitor is selected from (S)-1-[(3-hydroxy-1-adamantyl)amino]acetyl-2-cyano-pyrrolidine and (S)-1-(2-[5-cyanopyridin-2-yl)amino]ethyl-aminoacetyl)-2-cyano-pyrrolidine, and the further antidiabetic compound is selected from the group consisting of nateglinide, repaglinide, metformin, rosiglitazone, pioglitazone, troglitazone, glisoxepid, glyburide, glibenclamide, acetohexamide, chloropropamide, glibornuride, tolbutamide, tolazamide, glipizide, carbutamide, gliquidone, glyhexamide, phenbutamide, tolcyclamide, glimepiride and gliclazide, or the pharmaceutically acceptable salt of such a compound.

By the term “treatment” is understood the management and care of a patient for the purpose of combating the disease, condition, or disorder.

The term “prevention” means prophylactic administration of the combination to healthy patients to prevent the outbreak of the conditions mentioned herein. Moreover, the term “prevention” means prophylactic administration of such combination to patients being in a pre-stage of the conditions, especially diabetes, to be treated.

The term “delay of progression” used herein means administration of the combination, such as a combined preparation or pharmaceutical composition, to patients being in a pre-stage of the condition, especially diabetes, to be treated in which patients a pre-form of the corresponding condition is diagnosed.

The structure of the active agents identified by code nos., generic or trade names may be taken from the actual edition of the standard compendium “The Merck Index” or from databases, e.g., Patents International, e.g., IMS World Publications. The corresponding content thereof is hereby incorporated by reference. Any person skilled in the art is fully enabled to identify the active agents and, based on these references, likewise enabled to manufacture and test the pharmaceutical indications and properties in standard test models, both in vitro and in vivo.

The compounds to be combined can be present as pharmaceutically acceptable salts. If these compounds have, for example, at least one basic center, they can form acid addition salts. Corresponding acid addition salts can also be formed having, if desired, an additionally present basic center. The compounds having an acid group (for example COOH) can also form salts with bases. For example, the compounds to be combined can be present as a sodium salt, as a maleate or as a dihydrochloride. The active ingredient or a pharmaceutically acceptable salt thereof may also be used in form of a hydrate or include other solvents used for crystallization.

Examples of PPARα compounds, include but are not limited to, fenofibrate, micronized fenofibrate, bezafibrate, gemfibrazil and ciprofibrate, or a pharmaceutically acceptable salt of such a compound, and will be referred to hereinafter as COMBINATION PARTNER OF THE INVENTION. These PPARα compounds are disclosed in The Merck Index, 13^{th} Edition, (2001), the contents of which is hereby incorporated by reference in its entirety as if set forth in full herein.

Thus in a first aspect, the present invention concerns a combination, such as a combined preparation or fixed combination, respectively, which comprises a dipeptidylpeptidase-IV (DPP-IV) inhibitor and at least one peroxisome proflierator-activated receptor α (PPARα).

Preferably the invention concerns a combination for simultaneous, separate or sequential use Preferably the combination is a pharmaceutical combination-comprising a DPP-IV inhibitor in free or pharmaceutically acceptable salt form, and at least one further PPARα compound or the pharmaceutically acceptable salt of such a compound and optionally at least one pharmaceutically acceptable carrier; for simultaneous, separate or sequential use.

Preferably the DPP-IV inhibitor is selected from
(S)-1-[(3-hydroxy-1-adamantyl)amino]acetyl-2-cyano-pyrrolidine; and
(S)-1-{2-[5-cyanopyridin-2-yl)amino]ethyl-aminoacetyl}-2-cyano-pyrrolidine,
   in free or pharmaceutically acceptable salt form, and the further PPARα compound is selected from the group consisting of fenofibrate, micronized fenofibrate, bezafibrate, gemfibrazil and ciprofibrate, or the pharmaceutically acceptable salt of such a compound. Preferably the pharmaceutical combination is a combined preparation or a fixed combination. Preferably the pharmaceutical combination is a combined preparation for simultaneous, separate or sequential use.

A combined preparation which comprises a DPP-IV inhibitor in free or pharmaceutically acceptable salt form and at least one further COMBINATION PARTNER OF THE INVENTION and optionally at least one, i.e., one or more, e.g., two, pharmaceutically acceptable carrier for simultaneous, separate or sequential use is especially a “kit of parts” in the sense that the components, a DPP-IV inhibitor in free or pharmaceutically acceptable salt form and at least one further COMBINATION PARTNER OF THE INVENTION, can be dosed independently or by use of different fixed combinations with distinguished amounts of the components, i.e., at different time points or simultaneously. The parts of the kit of parts can then, e.g., be administered simultaneously or chronologically staggered, that is at different time points and with equal or different time intervals for any part of the kit of parts. Preferably, the time intervals are chosen such that the effect on the treated disease or condition in the combined use of the parts is larger than the effect which would be obtained by use of only any one of the components.

The present invention thus also relates to a kit of parts comprising

(a) an amount of a DPP IV inhibitor or a pharmaceutically acceptable salt thereof in a first unit dosage form;

(b) an amount of at least one PPARα compound or the pharmaceutically acceptable salt thereof,

in the form of two or three or more separate units of the components (a) and (b).

Preferably the DPP-IV inhibitor is selected from
(S)-1-[(3-hydroxy-1-adamantyl)amino]acetyl-2-cyano-pyrrolidine; and
(S)-1-{2-[5-cyanopyridin-2-yl)amino]ethyl-aminoacetyl}-2-cyano-pyrrolidine, and the further PPARα compound is selected from the group consisting of fenofibrate, micronized fenofibrate, bezafibrate, gemfibrazil and ciprofibrate.

The invention furthermore relates to a commercial package comprising the combination according to the present invention together with instructions for simultaneous, separate or sequential use.

Preferably, there is at least one beneficial effect, e.g., a mutual enhancing of the effect of a DPP-IV inhibitor in free or pharmaceutically acceptable salt form, and at least one further COMBINATION PARTNER OF THE INVENTION, additional advantageous effects, less side effects, a combined therapeutical effect in a non-effective dosage of one or each of the components, and especially a synergism, e.g., a more than additive effect, between a DPP-IV inhibitor in free or pharmaceutically acceptable salt form, and at least one further COMBINATION PARTNER OF THE INVENTION.

The nature of conditions mediated by DPP-IV, especially diabetes, conditions of impaired fasting plasma glucose, and IGT, is multifactorial. Under certain circumstances, drugs with different mechanisms of action may be combined. However, just considering any combination of drugs having different mode of action but acting in the similar field does not necessarily lead to combinations with advantageous effects.

All the more surprising is the experimental finding that the combined administration of a DPP-IV inhibitor and at least one further COMBINATION PARTNER OF THE INVENTION results not only in a beneficial, especially a synergistic, therapeutic effect but also in additional benefits resulting from combined treatment such as a surprising prolongation of efficacy, a broader variety of therapeutic treatment and surprising beneficial effects on diseases and conditions associated with diabetes, e.g., less gain of weight or less cardiovascular side effects.

The term “synergistic” shall mean that the drugs, when taken together, produce a total joint effect that is greater than the sum of the effects of each drug when taken alone.

The diseases, disorders or conditions associated to diabetes, particularly type 2 diabetes mellitus, includes but are not limited to diabetic nephropathy, diabetic retinopathy and diabetic neuropathy, macular degeneration, coronary heart disease, myocardial infarction, diabetic cardiomyopathy, myocardial cell death, coronary artery diseases, peripheral arterial disease, stroke, limb ischemia, vascular restenosis, foot ulcerations, endothelial dysfunction and/or atherosclerosis.

Further benefits are that lower doses of the individual drugs to be combined according to the present invention can be used to reduce the dosage, for example, that the dosages need not only often be smaller but are also applied less frequently, or can be used in order to diminish the incidence of side effects. This is in accordance with the desires and requirements of the patients to be treated.

It can be shown by established test models and especially those test models described herein that the combination of a DPP-IV inhibitor, especially (S)-1-{2-[5-cyanopyridin-2-yl)amino]ethyl-aminoacetyl}-2-cyano-pyrrolidine (DPP728) or (S)-1-[(3-hydroxy-1-adamantyl)amino]acetyl-2-cyano-pyrrolidine (LAF237), and at least one further COMBINATION PARTNER OF THE INVENTION results in a more effective prevention or preferably treatment of conditions mediated by DPP-IV, in particular diabetes, especially type 2 diabetes mellitus, conditions of impaired fasting plasma glucose, and conditions of IGT.

The person skilled in the pertinent art is fully enabled to select a relevant animal test model to prove the hereinbefore and hereinafter indicated therapeutic indications and beneficial effects. The pharmacological activity may, for example, be demonstrated following essentially an in vivo test procedure in mice or in a clinical study as described hereinafter.

In Vivo Test in Rats for Blood Glucose Control

Male Zucker fa/fa rats (Charles River, Pa.) fed standard rodent chow were housed individually in a room with a reverse light cycle (8:00 pm-8:00 am). The rats, 9-11 weeks of age, were cannulated in the right jugular vein 5-7 days prior to initiation of dosing. The animals were orally dosed once a day for 21 days with vehicle solution (0.5% methylcellulose; n=6), test compounds alone [NVP-LAF237 (10 umole/kg; n=7) or micronized fenofibrate (100 mg/kg; n=8)] or the combination (NVP-LAF237 and micronized fenofibrate). On the 21^{st} day, oral glucose tolerance test was performed. The rats were fasted for 18 hours before the test. Test compounds were given immediately after the baseline sample was taken at 15 minutes prior to glucose load. Glucose at 1 mg/kg was orally dosed to animals and blood samples were taken at 0, 5, 10, 15, 20, 30, 45, 60 and 90 minutes after dose. The blood samples were immediately transferred into chilled tubes (containing EDTA and trasylol solution) and centrifuged. Plasma samples were isolated and stored at −20° C. for the future determination of glucose, insulin, DPP-IV, fatty acid and triglyceride concentrations. Data were expressed as mean±SEM for each group. Two-way ANOVA analysis was performed. Our results clearly show;

the OGTT glucose excursion improvement in animals which were orally dosed once a day for 21 days with vehicle solution (0.5% methylcellulose; n=6), test compounds alone [NVP-LAF237 (10 mmole/kg; n=7) or micronized fenofibrate (100 mg/kg; n=8)] or the combination (NVP-LAF237 and micronized fenofibrate). The plasma glucose AUC (air under the curve) is reduced by 18% after administration of LAF237 alone, 7% after administration of micronized fenofibrate alone and 33% after administration of the combination LAF237 and micronized fenofibrate.

the OGTT insulin sensitivity improvement (by decreasing insulin resistance) in animals which were orally dosed once a day for 21 days with vehicle solution (0.5% methylcellulose; n=6), test compounds alone [NVP-LAF237 (10 mmole/kg; n=7) or micronized fenofibrate (100 mg/kg; n=8)] or the combination (NVP-LAF237 and micronized fenofibrate).

the reduction in body weight gain in animals which were orally dosed once a day for 21 days with vehicle solution (0.5% methylcellulose; n=6), test compounds alone [NVP-LAF237 (10 mmole/kg; n=7) or micronized fenofibrate (100 mg/kg; n=8)] or the combination (NVP-LAF237 and micronized fenofibrate).

The combination treatment of NVP-LAF237 and micronized fenofibrate significantly improved OGTT glucose, improved insulin sensitivity and reduced body weight gain as compared to vehicle or either test compound alone.

Clinical Double-Blind, Randomized, Parallel-Group Study in Subjects with Type 2 Diabetes Mellitus Inadequately Controlled on Diet Alone

This study proves, in particular, the benefits of the claimed combined preparation or pharmaceutical composition, respectively. The beneficial effects on conditions mediated by DPP-IV, in particular type 2 diabetes mellitus can be determined directly through the results of this study or by changes in the study design which are known as such to a person skilled in the art.

The study is, in particular, suitable to compare the effects of monotherapy with a COMBINATION PARTNER OF THE INVENTION with those of a combination of DPP-IV inhibitor plus one of these compounds on glycemic control.

Subjects with a diagnosis of type 2 diabetes mellitus who have not achieved near normoglycemia (HbA_{1c}<6.8%) on diet only are chosen for this trial. The effects on glycemic control achieved with DPP-IV monotherapy, monotherapy with one COMBINATION PARTNER OF THE INVENTION, and the combination therapy of DPP-IV plus one COMBINATION PARTNER OF THE INVENTION are determined in this study after 24 weeks with the control achieved on placebo, all subjects continuing with the same diet as in the period before treatment. Measures of glycemic control are validated surrogate endpoints for the treatment of diabetes. HbA_{1c} is the single most reliable measurement for assessing glycemic control (see Goldstein et al., “Tests of Glycemia in Diabetes”, Diabetes Care, Vol. 18, No. 6, pp. 896-909 (1995)) and is the primary response variable in this study. Since glycosylation of hemoglobin is determined by the glucose concentration at the time each red blood cell is made, HbA_{1c} provides an estimate of mean blood glucose for the previous three months.

The subjects are then separated into four treatment groups for the 24-week double-blind study (period II) as depicted in Tables 1-3 for the case that LAF237 is chosen as the DPP-IV inhibitor and one of the drugs comprising the micronized fenofibrate, bezafibrate or gemfibrazoil is chosen as the combination partner.

Examples for Combinations to be Administered

TABLE 1
LAF 237 Plus Micronized Fenofibrate

LAF 237 10 mg* + micronized fenofibrate placebo**
Micronized fenofibrate 200 mg** + LAF237 placebo*
LAF237 10 mg* + micronized fenofibrate 200 mg**
LAF237 placebo* + micronized fenofibrae placebo**
*Administered before breakfast, lunch and dinner.
**Administered once daily with breakfast.

TABLE 2
LAF237 Plus Bezafibrate

LAF237 10 mg* + bezafibrate placebo**
Bezafibrate 400 mg** + LAF237 placebo*
LAF237 10 mg* + bezafibrate 400 mg**
LAF237 placebo* + bezafibrate placebo**
*Administered before breakfast, lunch and dinner.
**Administered once daily with breakfast.

TABLE 3
LAF237 Plus Gemfibrazoil

LAF237 10 mg* + gemfibrazoil placebo**
Gemfibrazoil 600 mg** + LAF237 placebo*
LAF237 10 mg* + gemfibrazoil 600 mg**
LAF237 placebo* + gemfibrazoil placebo**
*Administered before breakfast, lunch and dinner.
**Administered twice a day with breakfast amd dinner.

LAF237 tablets contain either 10 mg of the compound or matching placebo. Micronied fenofibrate tablets contain either 200 mg or matching placebo. Micronized fenofibrate 200 mg tablets, bezafibrate 400 mg tablets and gemfibrazoil 600 mg tablets can be purchased commercially and over-encapsulated to match the corresponding placebo capsules. In another very preferred embodiment, the inventions concerns combinations as described in the above tables containing 50 mg of LAF237.

The subjects are then separated into four treatment groups for the 24-week double-blind study (period II) as depicted in Table 1. Approximately 170 subjects are randomized per treatment group. The total study duration including the run-in period for each subject is 28 weeks. Statistical analysis can be carried out by methods known in the art.

The subject is advised not to take the morning dose of study medication or eat breakfast on the day of a scheduled study visit. The morning dose is administered by site personnel after the collection of all fasting laboratory samples and completion of all study procedures. Visits are scheduled to be performed at 2 week intervals during period I, and 4-8 week intervals during period II. Subjects have fasted for at least 7 hours at the time of each visit. All blood samples for laboratory evaluations are drawn between 7:00 AM and 10:00 AM. All tests are conducted in accordance with Good Laboratory Practice principles following procedures known in the art.

HbA_{1c} is measured by High Performance Liquid Chromatography (HPLC) using the ion-exchange method on a Bio-Rad Diamat analyzer. A back-up affinity method are used if hemoglobin variants or hemoglobin degradation peaks are observed.

Further parameters to be determined are fasting plasma glucose (FPG), fasting lipids (total, high density lipoprotein (HDL)- and low density lipoprotein (LDL)-cholesterol and triglycerides) and body weight. FPG will be measured using the hexokinase method and LDL-cholesterol will be calculated using the Friedewald formula if triglycerides are <400 mg/dL (4.5 mmol/L).

Various parameters of the study described above can be modified, e.g., in order to optimize the dosage for special diseases or indications mentioned herein, to cope with tolerability problems during the study or to obtain similar or identical results with less efforts. For example, a different subject population can be involved in such a clinical trial, e.g.; subjects with a diagnosis of type 2 diabetes mellitus who have achieved near normoglycemia (HbA_{1c}<6.8%) on diet alone, subjects with diseases other than diabetes mellitus, e.g., other metabolic disorders, or subjects selected by other criteria, such as age or sex; the subject number can be decreased, e.g., to a number of between 70 and 150, especially 100 or 120, subjects per treatment group; treatment groups (listed exemplary in Table 1) can be deleted, i.e., for example to carry out a study with a comparison of the combination of a DPP-IV inhibitor and at least one further COMBINATION PARTNER OF THE INVENTION versus a DPP-IV inhibitor alone; the term of the placebo run-in period (period 1) can be changed, i.e., it can be extended, shortened or deleted; the visit schedule can be extended, e.g., to every 10, 12 or 14 weeks; the visit instructions can be changed, e.g., the instruction that blood samples for laboratory evaluations have to be drawn between 7:00 AM and 10:00 AM; HbA_{1c} can be determined by other means; or one or more of the parameters to be determined during the study mentioned above, e.g., FPG or fasting lipids, can be deleted or the determination of additional parameters (see below) can be added.

Additional parameters can be determined in the course of the study, e.g., by additional tests. Such additional tests can comprise the analysis of body liquids in order to determine amounts or numbers for parameters, such as those listed below, and can serve, e.g., the purpose of determining the tolerability of the administered active ingredients:
determination of hematocrit and hemogloblin, platelet count, erythrocyte count, total and differential leukocyte count, e.g., basophils, eosinophils, lymphocytes, monocytes, segmented neutrophils and total neutrophils;
determination of albumin, alkaline phosphatase, alanine amino transferase, e.g., serum glutamic pyruvic transaminase, aspartate amino transferase, e.g., serum glutamic oxaloacetic transaminase, blood urea nitrogen or urea, bicarbonate, calcium, chloride, total creatine phosphokinase (CPK), creatine phosphokinase muscle-brain fraction isoenzyme (if CPK is elevated), direct bilirubin, creatinine, γ-glutamyl transferase, lactate dehydrogenase, potassium, sodium, total bilirubin, total protein and uric acid in the blood;
determination of bilirubin, glucose, ketones, pH, protein and specific gravity in the subjects urine; and
determination of body weight, blood pressure, e.g., systolic and diastolic, after 3 minutes sitting, and radial pulse (after 3 minutes sitting).

The results of the studies show that the combination according to the present invention can be used for the prevention and preferably the treatment of conditions mediated by DPP-IV or PPARα, in particular type 2 diabetes mellitus and dyslipidemia. The combination of the present invention can also be used for the prevention and preferably the treatment of other condition mediated by DPP-IV or PPARα.

Furthermore, in a number of combinations as disclosed herein the side effects observed with one of the components surprisingly do not accumulate on application of the combination.

Preferably, the jointly therapeutically effective amounts of a DPP-IV inhibitor in free or pharmaceutically acceptable salt form and PPARα compound are administered simultaneously or sequentially in any order, separately or in a fixed combination.

The condition mediated by DPP-IV or PPARα is preferably selected from the group consisting of diabetes, impaired fasting plasma glucose, impaired glucose tolerance, metabolic acidosis, ketosis, arthritis, obesity, dyslipidemia and osteoporosis.

Very preferably, the condition mediated by DPP-IV is type 2 diabetes mellitus and the condition mediated by PPARα is dyslipidemia.

It is one objective of this invention to provide a pharmaceutical composition comprising a quantity, which is jointly therapeutically effective against conditions mediated by DPP-IV or PPARα, in particular diabetes, more especially type 2 diabetes mellitus, dyslipidemia, conditions of impaired fasting plasma glucose, and conditions of IGT, of a DPP-IV inhibitor: (i) or a pharmaceutically acceptable salt thereof; and (ii) at least one further COMBINATION PARTNER OF THE INVENTION and at least one pharmaceutically acceptable carrier.

The pharmaceutical compositions according to the invention can be prepared in a manner known per se and are those suitable for enteral, such as oral or rectal, and parenteral administration to mammals (warm-blooded animals), including man, comprising a therapeutically effective amount of the pharmacologically active compound, alone or in combination with one or more pharmaceutically acceptable carries, especially suitable for enteral or parenteral application.

The novel pharmaceutical preparations contain, for example, from about 10% to about 100%, e.g., 80% or 90%, preferably from about 20% to about 60%, of the active ingredient. Pharmaceutical preparations according to the invention for enteral or parenteral administration are, e.g., those in unit dose forms, such as sugar-coated tablets, tablets, capsules or suppositories and furthermore ampoules. These are prepared in a manner known per se, e.g., by means of conventional mixing, granulating, sugar-coating, dissolving or lyophilizing processes. Thus, pharmaceutical preparations for oral use can be obtained by combining the active ingredient with solid carriers, if desired granulating a mixture obtained, and processing the mixture or granules, if desired or necessary, after addition of suitable excipients to give tablets or sugar-coated tablet cores.

In this composition, components (i) and (ii) can be administered together, one after the other or separately in one combined unit dose form or in two separate unit dose forms. In one preferred embodiment of the invention, the unit dose form is a fixed combination. In a fixed combination the components (i) and (ii) are administered in the form of a single galenic formulation, e.g., a single tablet or a single infusion.

A further aspect of the present invention is the use of a pharmaceutical composition comprising a DPP-IV inhibitor and at least one further COMBINATION PARTNER OF THE INVENTION, in each case in free form or in form of a pharmaceutically acceptable salt thereof for the preparation of a pharmaceutical preparation for the prevention or treatment of conditions mediated by DPP-IV or PPARα, in particular diabetes, more especially type 2 diabetes mellitus, conditions of impaired fasting plasma glucose, dyslipidemia and conditions of IGT.

A therapeutically effective amount of each of the components of the combination of the present invention may be administered simultaneously or sequentially and in any order, and the components may be administered separately or as a fixed combination. For example, the method of treatment of the invention may comprise: (i) administration of a DPP-IV inhibitor in free or pharmaceutically acceptable salt form; and (ii) administration of at least one further COMBINATION PARTNER OF THE INVENTION simultaneously or sequentially in any order, in jointly therapeutically effective amounts, preferably in synergistically effective amounts, e.g., in daily dosages corresponding to the ratios described herein.

The corresponding active ingredient or a pharmaceutically acceptable salt thereof may also be used in form of a hydrate or include other solvents used for crystallization.

The invention relates in particular to a commercial package comprising jointly therapeutically effective amounts of a DPP-IV inhibitor, in free or pharmaceutically acceptable salt form, and at least one further COMBINATION PARTNER OF THE INVENTION together with instructions for use thereof in the treatment of conditions mediated by DPP-IV or PPARα, in particular diabetes, more especially type 2 diabetes mellitus, conditions of impaired fasting plasma glucose, dyslipidemia and conditions of IGT.

A further aspect of the present invention is a method of treating a condition mediated by DPP-IV or PPARα, in particular type 2 diabetes mellitus and dyslipidemia, comprising administering to a warm-blooded animal in need thereof jointly therapeutically effective amounts of a DPP-IV inhibitor in free or pharmaceutically acceptable salt form, and at least one further COMBINATION PARTNER OF THE INVENTION. Preferably, in this method of treating the active ingredients are administered simultaneously or sequentially in any order, separately or in a fixed combination. In one preferred embodiment of such method the jointly therapeutically effective amounts of a DPP-IV inhibitor in free or pharmaceutically acceptable salt form and at least one further COMBINATION PARTNER OF THE INVENTION are provided as a combined preparation.

Furthermore, the present invention provides a method of treating conditions of impaired glucose tolerance, dyslipidemia and impaired fasting plasma glucose comprising administering to a warm-blooded animal in need thereof jointly therapeutically effective amounts of a DPP-IV inhibitor in free or pharmaceutically acceptable salt form, and the COMBINATION PARTNER OF THE INVENTION.

The present invention furthermore concerns;

1) A combination according to the present invention for use as a medicament.

2) The use of a DPP-IV inhibitor in free or pharmaceutically acceptable salt form in combination with at least one further COMBINATION PARTNER OF THE INVENTION for the manufacture of a medicament for the treatment a condition mediated by DPP-IV or PPARα, in particular diabetes, more especially type 2 diabetes mellitus, conditions of IGT, conditions of impaired fasting plasma glucose, metabolic acidosis, ketosis, arthritis, obesity, dyslipidemia and osteoporosis.

3) The use of a DPP-IV inhibitor selected from

(S)-1-[(3-hydroxy-1-adamantyl)amino]acetyl-2-cyano-pyrrolidine; and
(S)-1-[(2-[5-cyanopyridin-2-yl)amino]ethyl-aminoacetyl]-2-cyano-pyrrolidine,
   or pharmaceutically acceptable salt form in combination with at least one further PPARα compound selected from the group consisting of fenofibrate, micronized fenofibrate; bezafibrate, gemfibrazil and ciprofibrate, or the pharmaceutically acceptable salt of such a compound INVENTION for the manufacture of a medicament for the treatment a condition mediated by DPP-IV or PPARα, in particular diabetes, type 2 diabetes, impaired glucose tolerance, obesity and dyslipidemia.
   4) The use of a (S)-1-[(3-hydroxy-1-adamantyl)amino]acetyl-2-cyano-pyrrolidine
   or pharmaceutically acceptable salt form in combination with at one further PPARα compound selected from the group consisting of fenofibrate, micronized fenofibrate, bezafibrate, gemfibrazil and ciprofibrate, or the pharmaceutically acceptable salt of such a compound INVENTION for the manufacture of a medicament for the treatment a condition mediated by DPP-IV or PPARα, in particular type 2 diabetes, impaired glucose tolerance, obesity and dyslipidemia.
   5) Use of a combination according to the present invention for the manufacture of a medicament for the treatment a condition mediated by DPP-IV or PPARα, in particular diabetes, more especially type 2 diabetes mellitus, conditions of IGT, conditions of impaired fasting plasma glucose, metabolic acidosis, ketosis, arthritis, obesity, dyslipidemia and osteoporosis.

The dosage range of the combination of a DPP-IV inhibitor and at least one further COMBINATION PARTNER OF THE INVENTION to be employed depends upon factors known to the person skilled in the art including species of the warm-blooded animal, body weight and age, the nature and severity of the condition to be treated, the mode of administration and the particular substance to be employed. Unless stated otherwise herein, the DPP-IV inhibitor and at least one further COMBINATION PARTNER OF THE INVENTION are preferably divided and administered from one to four times per day.

The weight ratio of the daily doses of DPP728 or LAF237 or a pharmaceutically acceptable salt thereof to at least one further COMBINATION PARTNER OF THE INVENTION may vary within wide limits depending, in particular, on the needs of the warm-blooded animal treated.

All references, including U.S., World and EP Patents and applications referred to herein are hereby incorporated by reference in their entirety as if set forth in full herein.

What is claimed is:

1. Combination comprising a dipeptidylpeptidase-IV (DPP-IV) inhibitor and at least one peroxisome proflierator-activated receptor α (PPARα).

2. A pharmaceutical composition comprising a DPP-IV inhibitor in free or pharmaceutically acceptable salt form, and at least one further PPARα compound or the pharmaceutically acceptable salt of such a compound and optionally at least one pharmaceutically acceptable carrier; for simultaneous, separate or sequential use.

3. The pharmaceutical composition according to claim 1, wherein the further PPARα compound is selected from the group consisting of fenofibrate, micronized fenofibrate, bezafibrate, gemfibrazil and ciprofibrate or the pharmaceutically acceptable salt of such a compound.

4. The pharmaceutical composition according to claim 1, which is a fixed combination.

5. The pharmaceutical composition according to claim 1, which is a combined preparation.

6. The pharmaceutical composition according to claim 5 which is a combined preparation for simultaneous, separate or sequential use in the prevention, delay of progression or treatment of conditions mediated by DPP-IV or PPARα.

7. The combination according to claim 1 or a pharmaceutical composition according to any one of claims 2 to 6, wherein the DPP-IV inhibitor is a N-(N′-substituted glycyl)-2-cyanopyrrolidine of formula (I) 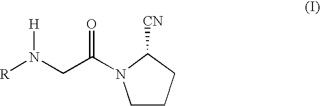
wherein R is:
a) R₁R₁ₐN(CH₂)ₘ—,
wherein
R₁ is a pyridinyl or pyrimidinyl moiety optionally mono- or, independently, di-substituted with lower alkyl, lower alkoxy, halogen, trifluoromethyl, cyano or nitro; or phenyl optionally mono- or, independently, di-substituted with lower alkyl, lower alkoxy or halogen;
R₁ₐ is hydrogen or (C₁₋₈)alkyl; and
m is 2 or 3;
b) (C₃₋₁₂)Cycloalkyl optionally mono-substituted in the 1-position with (C₁₋₃)hydroxyalkyl;
c) R₂(CH₂)ₙ—,
wherein either
R₂ is phenyl optionally mono- or independently di- or, independently, tri-substituted with lower alkyl, lower alkoxy, halogen or phenylthio optionally mono-substituted in the phenyl ring with hydroxymethyl; or is (C₁₋₈)alkyl; a [3.1.1]bicyclic carbocyclic moiety optionally mono- or pluri-substituted with (C₁₋₈)alkyl; a pyridinyl or naphthyl moiety optionally mono- or, independently, di-substituted with lower alkyl, lower alkoxy or halogen; cyclohexene; or adamantyl; and
n is 1-3; or
R₂ is phenoxy optionally mono- or, independently, di-substituted with lower alkyl, lower alkoxy or halogen; and
n is 2 or 3;
d) (R₃)₂CH(CH₂)₂—, wherein each R₃, independently, is phenyl optionally mono- or, independently, di-substituted with lower alkyl, lower alkoxy or halogen;
e) R₄(CH₂)ₚ—,
wherein
R₄ is 2-oxopyrrolidinyl or (C₂₋₄)alkoxy; and
p is 2-4;
f) isopropyl optionally mono-substituted in 1-position with (C₁₋₃)hydroxyalkyl;
g) R₅, wherein R₅ is indanyl, a pyrrolidinyl or piperidinyl moiety optionally substituted with benzyl, a [2.2.1]- or [3.1.1]bicyclic carbocyclic moiety optionally mono- or pluri-substituted with (C₁₋₈)alkyl, adamantyl or (C₁₋₈)alkyl optionally mono- or, independently, pluri-substituted with hydroxy, hydroxymethyl or phenyl optionally mono- or, independently, di-substituted with lower alkyl, lower alkoxy or halogen;
h) a substituted adamantyl
in free form or in acid addition salt form.

8. The combination according to claim 1 or a pharmaceutical composition according to any one of claims 2 to 6, wherein the DPP-IV inhibitor a compound of formula (I) which is selected from
(S)-1-[(3-hydroxy-1-adamantyl)amino]acetyl-2-cyano-pyrrolidine; and
(S)-1-{2-[5-cyanopyridin-2-yl)amino]ethyl-aminoacetyl}-2-cyano-pyrrolidine;
in free form or in acid addition salt form.

9. The combination according to claim 1 or a pharmaceutical composition according to any one of claims 2 to 6, wherein the DPP-IV inhibitor is selected from
(S)-1-[(3-hydroxy-1-adamantyl)amino]acetyl-2-cyano-pyrrolidine; and
(S)-1-{2-[5-cyanopyridin-2-yl)amino]ethyl-aminoacetyl}-2-cyano-pyrrolidine,
and the further PPARα compound is selected from the group consisting of fenofibrate, micronized fenofibrate, bezafibrate, gemfibrazil and ciprofibrate,
or the pharmaceutically acceptable salt of such a compound.

10. A method of treating a condition mediated by DPP-IV or PPARα comprising administering to a warm-blooded animal in need thereof jointly therapeutically effective amounts of a DPP-IV inhibitor in free or pharmaceutically acceptable salt form and at least one PPARα compound, or the pharmaceutically acceptable salts of such compounds.

11. The method of claim 10, wherein the condition is dyslipidemia.

12. The method of claim 10, wherein the condition is diabetes.

13. The method of claim 12, wherein the condition is type II diabetes.

14. The method of claim 10, wherein the condition is obesity.

15. A combination or pharmaceutical composition according to any one of the claims 1 to 9, for use as a medicament.

16. Use of a DPP-IV inhibitor in free or pharmaceutically acceptable salt form in combination with at least one further PPARα compound in free or pharmaceutically acceptable salt form, for the manufacture of a medicament for the treatment a condition mediated by DPP-IV or PPARα.

17. Use of a DPP-IV inhibitor selected from
(S)-1-[(3-hydroxy-1-adamantyl)amino]acetyl-2-cyano-pyrrolidine; and
(S)-1-{(2-[5-cyanopyridin-2-yl)amino]ethyl-aminoacetyl}-2-cyano-pyrrolidine,
in free or pharmaceutically acceptable salt form in combination with at least one further PPARα compound selected from the group consisting of fenofibrate, micronized fenofibrate, bezafibrate, gemfibrazil and ciprofibrate, or the pharmaceutically acceptable salt of such a compound for the manufacture of a medicament for the treatment a condition mediated by DPP-IV or PPARα.

18. Use of a pharmaceutical composition according to any one of claims 2 to 6 for the manufacture of a medicament for the treatment a condition mediated by DPP-IV or PPARα.

19. Use according to any one of claims 16 to 18, wherein the condition mediated by DPP-IV or PPARα, is selected from diabetes, type 2 diabetes mellitus, conditions of IGT, conditions of impaired fasting plasma glucose, metabolic acidosis, ketosis, arthritis, obesity, dyslipidemia and osteoporosis

20. Use according to any one of claims 16 to 18, wherein the condition mediated by DPP-IV or PPARα, is selected from type 2 diabetes, impaired glucose tolerance, obesity and dyslipidemia.

21. A commercial package comprising as active agents a combination of a DPP-IV inhibitor and a PPARα compound together with instructions for simultaneous, separate or sequential use thereof in the prevention, delay of progression or treatment of a condition mediated by DPP-IV or PPARα.

22. A kit of parts comprising
(a) an amount of a DPP IV inhibitor or a pharmaceutically acceptable salt thereof in a first unit dosage form;
(b) an amount of at least one PPARα compound or the pharmaceutically acceptable salt thereof,
in the form of two or three or more separate units of the components (a) and (b).

23. A kit of parts according to claim 22 or a commercial package according to claim 21, wherein the DPP-IV inhibitor is selected from
(S)-1-[(3-hydroxy-1-adamantyl)amino]acetyl-2-cyano-pyrrolidine; and
(S)-1-{2-[5-cyanopyridin-2-yl)amino]ethyl-aminoacetyl}-2-cyano-pyrrolidine, and the further PPARα compound is selected from the group consisting of fenofibrate, micronized fenofibrate, bezafibrate, gemfibrazil and ciprofibrate
